(19) United States
(12) Patent Application Publication
(10) Pub. No.: US 2026/0226216 A1
(43) Pub. Date: Aug. 6, 2026

(54) CURABLE URETHANE DI(METH)ACRYLATES

(71) Applicant: RAHN AG, Zürich (CH)

(72) Inventors: Roberto BIANCHI, Wettingen (CH); Anna DI GIANNI, Daettwil (CH); Andreas MOECK, Baden (CH); Reto WEDER, Neerach (CH)

(21) Appl. No.: 19/154,971

(22) PCT Filed: Feb. 7, 2024
(86) PCT No.: PCT/EP2024/053104

§ 371 Date: Aug. 8, 2025

(30) Foreign Application Priority Data
Feb. 10, 2023 (EP) 23155967.5

(51) Int. Cl. C08F 222/22; A61K 6/30; A61K 6/893; C07C 265/10; C09D 135/02

(52) U.S. Cl. CPC C08F 222/22; A61K 6/30; A61K 6/893; C07C 265/10; C09D 135/02

(57) ABSTRACT

The present invention relates to a curable urethane di(meth)acrylate comprising a diisocyanato component and a (meth)acrylate component, characterized in that the diisocyanato component consists of 50 to 75% by weight of 1,6-diisocyanatohexane (HDI), and at least one further diisocyanate, and the (meth)acrylate component consists of 50 to 100% by weight of hydroxypropyl-methacrylate (HPMA) and optionally at least one further methacrylate or acrylate component.

The present invention relates to curable urethane di(meth)acrylates, their use and preparation.

There are large numbers of (meth)acrylate copolymers that can be produced by bulk radical polymerization processing conditions where the resulting polymers generally display either good strength properties or good flexibility.

Urethane (meth)acrylate monomers and oligomers such as 1,6-bis(methacryloxy-2-ethoxycarbonylamino)-2,2,4(2,4,4)-trimethylhexane (UDMA) are used as components of photopolymer formulations. These compounds provide excellent mechanical properties (flexibility and strength typically imparted by the urethane group), low viscosity, better filler loading capacity, optical transparency, and low toxicity. Moreover, the presence of urethane functionalities, with their proton donor group, allows often to obtain further enhanced mechanical properties when used in composite applications.

TMDI is a mixture of 2,2,4- and 2,4,4-trimethyl-hexamethylene diisocyanate. Its use as diisocyanate reactant is key for guaranteeing both an easily manageable synthesis process and nice final oligomer properties. TMDI, is indeed a liquid compound at room temperature and its chemical structure, contributes to provide low viscosity and no crystallization tendencies to the final product.

One of the main problems associated with the use of TMDI is however its limited market availability.

Commercial alternatives to TMDI exist. Examples are 1,6-diisocyanatohexane (HDI), isophorone diisocyanate (IPDI), 4,4′-diisocyanatodicyclohexylmethane (H12MDI), 2,4-diisocyanatotoluene (TDI) and bis-(4-isocyanatophenyl) methane (MDI).

EP441383 discloses plastic lenses formed by curing a urethane poly(meth)acrylate which is an adduct of 2-hydroxyethyl (meth)acrylate (2-HEMA) or 2-hydroxypropyl (meth)acrylate (2-HPMA) with isophorone diisocyanate (INCI), 2,2,4-trimethylhexamethylene diisocyanate (TMDI), dicyclohexylmethane diisocyanate (H12MDI), xylylene diisocyanate, 1,3-bis(α,α-dimethylisocyanatomethyl)benzene, tolylene diisocyanate (TDI) or naphthalene diisocyanate (MDI).

CN 115 477 912 relates to the preparation and application of bio-based ultraviolet curable adhesive for the production of wet laminated inductors. The method allows the use of lignin as a raw material, which can reduce petrochemical products in the raw material, improve biochemical properties, and reduce carbon dioxide emissions in disguise. It can also adapt to faster production efficiency, reduce VOC emissions in the production process, make the environment more friendly, and the raw materials are more green.

Xu Yuling et al («A triethylene glycol dimethacrylate-free dental composite for reduced water-sorption and shrinkage, Journal of composite materials, vol. 52, No. 12, pages 1579-1588) disclose a study on new liquid urethane-based oligomers for dental composites, comparing them with commercial systems. Their findings show that these oligomers, especially when derivatized with varying methacrylates, lead to composites with reduced water sorption and solubility, less shrinkage, and enhanced mechanical strength compared to traditional resin-based composites.

CN 111 333 811 discloses a method for creating a UV-curable polyurethane acrylate resin with high wear resistance and toughness. This cost-effective and easy-to-produce resin is ideal, for wear-resistant products like phone shells and decorations. The method involves mixing 75 to 85% by weight of hydroxy acrylate, 5 to 10% by weight of a first isocyanates, 7 to 15% by weight of a second isocyanate, a catalyst, a polymerization inhibitor, and an antioxidant, then stirring at 65-75° C. until the isocyanate ion concentration is adequately reduced.

WO2019243339 UDMA variants prepared by reaction of
HEMA with 2,4-dimethylhexamethylene diisocyanate,
HEMA with hexamethylene diisocyanate
HEMA with isophorone diisocyanate
HEMA with 2,2,4,4-tetramethylhexamethylene diisocyanate
HPMA with 2,2,4-trimethylhexamethylene diisocyanate
HPMA with 2,4,4-trimethylhexamethylene diisocyanate
HPMA with 2,4-dimethylhexamethylene diisocyanate
HPMA with hexamethylene diisocyanate
HPMA with isophorone diisocyanate
HPMA with 2,2,4,4-tetramethylhexamethylene diisocyanate and
the variants which can be produced by reaction of the abovementioned diisocyanates with hydroxyethylacrylate (HEA) and hydroxypropylacrylate (HPA).

U.S. Pat. No. 4,861,853 relates to isocyanate functional polymers useful as crosslinking agents. One example of an isocyanate functional unsaturated monomer is the reaction product of isophorone diisocyanate (IPDI) with hydroxyethyl (meth)acrylate (HEMA).

However, said UDMA alternatives have significant drawbacks. Some of them are high viscous or even solid at room temperature, others show crystallization or decreased mechanical properties.

The object of the present invention was therefore to provide new UDMA alternatives with sufficient low viscosity and low crystallization tendencies.

The problem is solved by the urethane di(meth)acrylates according to the present invention. Further preferred embodiments are subject of dependent claims 2 to 15.

It was shown that the curable urethane di(meth)acrylates according to the present invention have excellent mechanical properties as well as a low viscosity and a low tendency to form crystals. In fact, their mechanical properties are similar to the mechanical properties of UDMA. Thus, the curable urethane di(meth)acrylates according to the present invention are an excellent alternative to UDMA.

The curable urethane di(meth)acrylate according to the present invention comprises a diisocyanato component and a (meth)acrylate component and characterized in that
i. the diisocyanato component consists of 50 to 75% by weight of 1,6-diisocyanatohexane (HDI), and at least one further diisocyanate, and
ii. the (meth)acrylate component comprises 50 to 100% by weight of hydroxypropylmethacrylate (HPMA) and optionally at least one further methacrylate or acrylate.

Thus, the diisocyanato component comprised in the urethane di(meth)acrylates according to the present invention is a mixture of at least two different diisocyanates. The diisocyanato component always comprises 50 to 75% by weight of 1,6-diisocyanatohexane (HDI), the remainder being one or more further diisocyanates with the percentages of all diisocyanates adding up to 100%. Within the context of the present invention the expression “diisocyanate” stands for a monomer having two isocyanate groups.

The (meth)acrylate component consists either of hydroxypropylmethacrylate (HPMA) alone or comprises at least one further methacrylate or acrylate component with the percentages of all (meth)acrylates adding up to 100%. The term (meth)acrylate component encompasses acrylate monomers and methacrylate monomers.

Due to the unique combination of the diisocyanato component and the (meth)acrylate component, the urethane di(meth)acrylates according to the present invention have a low viscosity, that is, a viscosity of less than 150′000 mPas at 25° C., most preferably less than 50′000 mPas and ideally less than 35′000 mPas while at the same time showing no or only low crystallization tendencies. Such a viscosity allows easy handling of the urethane di(meth)acrylates according to the present invention.

Preferably, the at least one further diisocyanate is selected from the group consisting of isophorondiisocyanate (IPDI; CAS 4098-71-9), diisocynatodicyclohexylmethane (H12MDI; CAS 5124-30-1), toluol-2,4-diisocyanat (2,4-TDI, CAS 584-84-9), toluol-2,6-diisocyanat (2,6-TDI, CAS 91-08-7), diphenylmethane 4,4′-diisocyanate MDI (CAS 101-68-8), 1,5-diisocyanatopentane (also called 1,5-pentamethylene diisocyanate; PDI; CAS 4538-42-5) and tetramethylxylylene diisocyanate (TMXDI, CS 58067-42-8) or a mixture thereof (such as 20% 2,6-TDI+80% 2,4-TDI (CAS 26471-62-5).

In one embodiment of the present invention the (meth)acrylate component consists of hydroxypropyl methacrylate (HPMA) alone which results in composition having low crystallization tendencies.

In a further preferred embodiment of the present invention the (meth)acrylate component consists of mixture of several different (meth)acrylates, preferably of the two, three, four, five or six different (meth)acrylates. The at least one further methacrylate component or acrylate component is preferably selected from the consisting group of hydroxyethyl methacrylate (HEMA), hydroxyethyl acrylate (HEA), hydroxypropyl acrylate (HPA), hydroxybutyl acrylate (HBA) and hydroxybutyl methacrylate (HBMA). Possible combinations for example are HPMA and HEMA; HPMA and HEA; HPMA and HPA; HPMA and HBA; HPMA and HBMA; HPMA, HEMA and HEA; HPMA, HEMA and HPA; HPMA, HEMA and HBA; HPMA, HEMA and HBMA; HPMA, HEA and HPA; HPMA, HEA and HBA; HPMA, HPA and HBMA; HPMA, HPA and HBA; HPMA, HPA and HBMA; HPMA, HHBA and HBMA; HPMA, HEMA, HEA, and HPA; HPMA, HEMA, HEA, and HBA; HPMA, HEMA, HEA, and HBMA; HPMA, HEA, HPA, and HBA; HPMA, HEA, HPA, and HBMA; HPMA, HPA, HBA and HBMA; HPMA, HEMA, HEA, HPA and HBA; HPMA, HEMA, HEA, HPA and HBMA; HPMA, HEA, HPA, HBA and HBMA; HPMA, HEMA, HPA, HBA and HBMA; HPMA, HEMA, HEA, HBA and HBMA; HPMA, HEA, HEMA, HPA, HBA and HBMA;

whereby the (meth)acrylate component always comprises at least 50% by weight of HPMA. The percentages of all (meth)acrylates add up to 100%.

In one embodiment the (meth)acrylate component of the urethane di(meth)acrylate according to present invention consists of 60-95% by weight of hydroxypropylmethacrylate (HPMA), 5-40% by weight of hydroxyethyl methacrylate (HEMA), and optionally 0 to 10% by weight of an acrylate component selected from the group consisting of hydroxyethyl acrylate (HEA), hydroxypropyl acrylate (HPA) and hydroxybutyl acrylate (HBA) or a mixture thereof. The presence of an acrylate can positively influence the reactivity.

In one embodiment the (meth)acrylate component of the urethane di(meth)acrylate according to present invention consists of 5-35% by weight of hydroxyethyl methacrylate (HEMA) and 65-95% by weight of hydroxypropylmethacrylate (HPMA). By replacing part of the HEMA by HPMA the tendency to form crystals can be reduced.

In another embodiment the (meth)acrylate component of the urethane di(meth)acrylate according to present invention consists of 20-35% by weight of hydroxyethyl methacrylate (HEMA) and 65-80% by weight of hydroxypropylmethacrylate (HPMA). By replacing a higher part of HPMA by HEMA the tendency to form crystals can be further reduced.

In another embodiment the (meth)acrylate component of the urethane di(meth)acrylate according to present invention consists of 20-30% by weight of hydroxyethyl methacrylate (HEMA) and 70-80% by weight of hydroxypropyl methacrylate (HPMA). It has been shown that such a meth (acrylate) ratio in combination with the diisocyanato component according to the present invention results in products with almost no crystal formation.

In one embodiment the diisocyanato component of the urethane di(meth)acrylate according to the present invention comprises 60 to 65% by weight of 1,6-diisocyanatohexane (HDI). The regular long saturated chain of HDI results in low viscosity. However, it also favors the formation of crystals. Optimal results with regard to viscosity and crystallization could be obtained with a HDI content of 60 to 65% by weight of the diisocyanato component. In this embodiment of the present invention HDI can be combined with IPDI or with H12MDI or with a combination thereof.

Preferably, the diisocyanato component of the urethane di(meth)acrylate according to the present invention comprises 60 to 65% by weight of 1,6-diisocyanatohexane (HDI) and 35% to 40% by weight of isophorondiisocyanate (IPDI).

In another preferred embodiment, the diisocyanato component of the urethane di(meth)acrylate according to the present invention comprises 60 to 65% by weight of 1,6-diisocyanatohexane (HDI) and 30 to 35% by weight of 4,4′-diisocynatodicyclohexylmethane (H12MDI). Preferably, corresponding diisocyanato component consists of 5-35% by weight of hydroxyethyl methacrylate (HEMA) and 65-95% by weight of hydroxypropylmethacrylate (HPMA), most preferably 25-30% by weight HEMA and 70-75% by weight HPMA, resulting in urethane di(meth)acrylates having an optimal viscosity and a low tendency to form crystals.

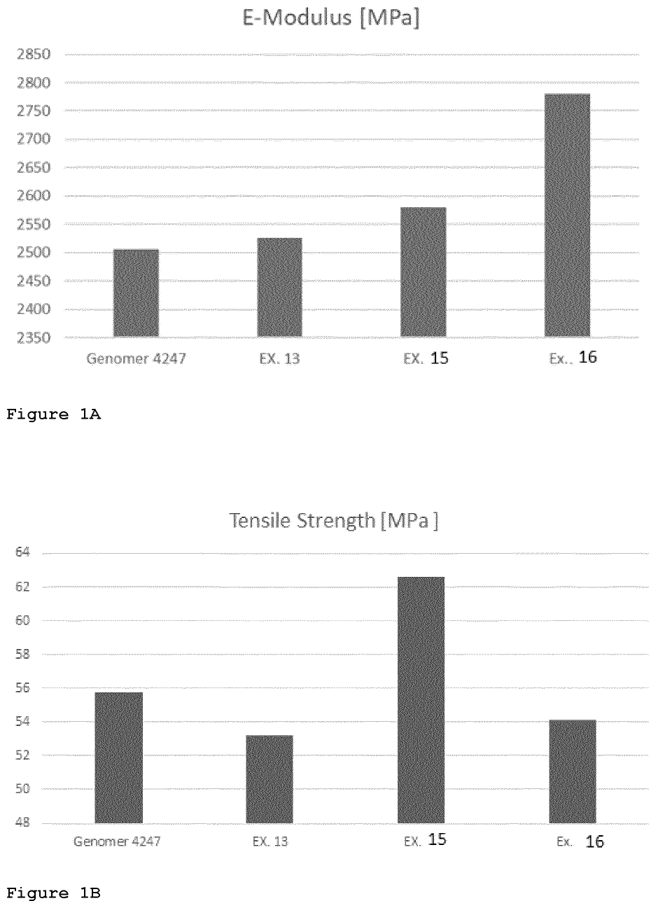

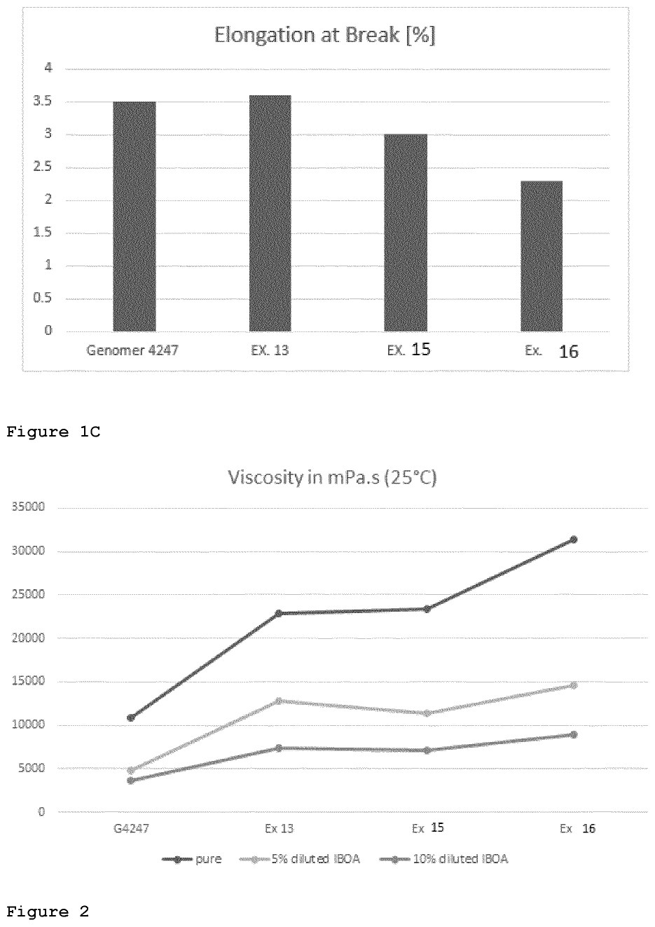

Urethane-di(meth)acrylates comprising a diisocyanato component consisting of 60 to 65% by weight of 1,6-diisocyanatohexane (HDI) and 35% to 40% by weight of isophorondiisocyanate (IPDI) or 30 to 35% by weight of 4,4′-diisocynatodicyclohexylmethane (H12MDI) and a (meth)acrylate component consists of 75 to 100% by weight of hydroxypropylmethacrylate (HPMA) and 0 to 25% by weight of hydroxyethyl methacrylate (HEMA) show excellent mechanical properties which are very similar to the mechanical properties of TMDI/HEMA (UDMA) (see FIGS. 1A to 1C). Furthermore, as shown in FIG. 2, the viscosity of the compositions according to the present invention can be further adjusted depending on the application.

A further aspect relates to a composition comprising the urethane di(meth)acrylate according to the present invention. The composition may comprise inorganic fillers or diluents.

In particular, the composition according to the present invention may comprise one or more diluents. Diluents reduce the viscosity of the urethane di(meth)acrylate for processing and—in case of reactive diluents—may become part material during its subsequent curing via copolymerization. Useful diluents include, for example: hydroxypropylmethacrylate (HPMA), hydroxybutylacrylate (HBA), isobornyl methacrylate (IBOMA) and acrylate (IBOA), 4-acryloylmorpholine (ACMO), tetra(ethylene glycol) diacrylate (TEGDA), 2-hydroxyethylacrylate (HEA) and methacrylate (HEMA), tetra(ethylene glycol) dimethacrylate (TEGDMA), cyclic trimethilolpropane formalacrylate (CTFA), diciclypentadienylacrylate (DCPA), 3-methyl-1,5-pentanediol diacrylate (MPDDA), tricyclodecane dimethanol diacrylate (TCDDA), trimethyl cyclohexylacrylate (TMCHA), glyceryl propoxy triacrylate (GPTA), hydroxyl pivalic acid neopentyl glycol diacrylate (HPNDA, 1,3-propanediol diacrylate and dimethacrylate, triethylenglycoldiacrylate and dimethacrylate, isobornylacrylate, 1,4-butanediol diacrylate and dimethacrylate, 1,5-pentanediol diacrylate and dimethacrylate, 1,6-hexanediol diacrylate and dimethacrylate, 1,7-heptanediol diacrylate and dimethacrylate, 1,8-octanediol diacrylate and dimethacrylate, trimethylolpropanetriol triacrylate and trimethacrylate, ethoxylated trimethylolpropanetriol triacrylate and trimethacrylate, neopentyl glycol diacrylate and dimethacrylate, tripropylene glycol diacrylate and dimethacrylate, pentaerythritol triacrylate and trimethacrylate, pentaerythritol tetraacrylate and tetramethacrylate, and the like.

A further aspect relates to the use of the composition according to the present invention for radiation curing applications. Radiation curing offers environmentally friendly and economic processing conditions. Within the context of the present invention the term radiation curing means for example electron beam radiation, ultraviolet radiation, visible light radiation or light emitting diode radiation.

A further aspect relates to the use of the composition according to the present invention for free-radical curing applications. Such a reaction is carried out in the presence of a free radical-generating catalyst which allows a fast curing and an excellent heat resistance. Suitable free radical generating catalysts include for example peroxide. Examples of peroxide catalysts include organo peroxides and hydroperoxides such as tert-butyl peroxyneodecanoate, benzoyl peroxide, dicumyl peroxide, methyl ethyl ketone peroxide, lauryl peroxide, cyclohexanone peroxide, t-butyl perbenzoate, t-butyl hydroperoxide, t-butylbenzene hydroperoxide, cumene hydroperoxide, t-butyl peroctoate, and the like. The composition according to the present invention may comprise 0.001 to 10 percent by weight of a free radical-generating catalyst.

The composition of the invention can—in general—be used for a multitude of purposes. Said liquid compositions are particularly suited for 3D printing, dental applications and nail gel applications.

Preferred 3D printing technologies are selected from the group consisting of stereolithography (SLA), direct light projection (DLP), material jetting, binder jetting and direct deposition inkjet.

Compositions comprising the urethane di(meth)acrylate according to the present invention are suitable for the preparation of polymers, adhesives and in particular dental materials, such as filling composites, adhesives and cements, e.g. fixing cements. Due to the low viscosity, either materials with a high filler content or else materials with a low viscosity become accessible. Particularly readily-flowing and thus easily processable composites, so-called “flowable” filling composites, can therefore be prepared. Furthermore, compositions containing fillers are particularly suitable as dental filling composites, cements and coating materials. Compositions are particularly preferred which only contain fillers with a maximum particle size of less than 600 nm. These are particularly suitable as dental cements.

The invention provides compositions urethane di(meth)acrylate for producing pigmented and non-pigmented paints and varnishes, powder coatings, printing inks, printing plates, adhesives, pressure sensitive adhesives, dental compositions, gel coats, photoresists, electroplating resists, etch resists, both liquid and dry films, solder resists, resists to manufacture color filters, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, color proofing systems, glass fiber cable coatings, screen printing stencils, for producing three-dimensional objects by means of stereo-lithography or material jetting, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board.

In a preferred embodiment of the invention the composition according to the present invention is used for producing a three-dimensional object by 3d printing, preferably by means of stereolithography (SLA), direct light projection (DLP), material jetting, binder jetting and direct deposition inkjet. Three dimensional objects for the purpose of the invention are not limited but, for example, a toy, a medical instrument, an electronic part, an automotive part, a motorcycle part, a train part, an aircraft part, another industrial part, a dental material such as an artificial tooth and a dental corrective devices, a jewelry, a hearing aid, an earpiece, a positive die for lost wax mold to produce thereof, and a prototype for design verification or performance check for development and manufacture of industrial products.

The composition according to the present invention is preferably present as single-component systems, i.e. in the form of a mixture which contains all the constituents of the composition. As initiator they contain exclusively a photoinitiator and can be cured by irradiation with light.

The composition according to the present invention is present as dual-component systems. The curing of the dual-curing compositions can be activated by mixing the catalyst and accelerator pastes. The composition is adjusted such that it still remains processable for a few minutes after the pastes are mixed (so-called processing time), but cures rapidly after the processing. The processing and curing times can be adjusted primarily through the type and concentration of (hydro) peroxide, thiourea derivative and optionally by the addition of further components such as a transition metal redox catalyst and inhibitor.

A further aspect relates to method for preparing the urethane di(meth)acrylate according to the present invention, i.e. to a curable urethane di(meth)acrylate comprising a diisocyanato component and a (meth)acrylate component, wherein
i. the diisocyanato component consists of 50 to 75% by weight of 1,6-diisocyanatohexane (HDI), and at least one further diisocyanate, and
ii. the (meth)acrylate component consists of 50 to 100% by weight of hydroxypropylmethacrylate (HPMA) and optionally at least one further methacrylate or acrylate component.

The method may involve the following steps:
(i) Preparing the diisocyanato component by mixing HDI the at least one further diisocyanate to obtain a mixture of all diisocyanates, whereby the HDI content of all diisocyanates is 50 to 75% by weight,
(ii) Adding either
   a. HPMA, or
   b. a mixture of two or more (meth)acrylates, whereby one of said two or more (meth)acrylates present is hydroxypropyl-methacrylate (HPMA) constituting a minimum of 50% by weight of the (meth)acrylate content, for example a mixture of HPMA and HEMA; HPMA and HEA; HPMA and HPA; HPMA and HBA; HPMA, HEMA and HEA; HPMA, HEMA and HPA; HPMA, HEMA and HDA; HPMA, HDA and HPA; HPMA, HEMA, HEA and HPA, HPMA, HEMA, HEA and HBA, HPMA, HEMA, HEA, HBA and HPA, or
   c. the two more or (meth)acrylates sequentially, whereby one of said two or more (meth)acrylates present is hydroxypropyl-methacrylate (HPMA) constituting a minimum of 50% by weight of the (meth)acrylate content, i.e., adding first a first (meth)acrylate followed by a second (meth)acrylate, followed by an optional third (meth)acrylate, followed by an optional forth (meth)acrylate,
(iii) reacting the resulting mixture optionally in the presence of a catalyst to obtain the urethane di(meth)acrylate according to the present invention.

Due to their differences in reactivity, the diisocyanates are preferably premixed before adding the (meth)acrylate component. The (meth)acrylates can be premixed before adding them to the diisocyanate component. Alternatively, they can be subsequently added. The exothermic reaction can be controlled by the addition speed of the (meth)acrylate component.

Alternatively, the urethane di(meth)acrylate according to the present invention can be prepared by a method involving the following steps:

(i) Filling HDI in a reactor

(ii) Adding HPMA to HDI and mixing them together, thus, HDI is already present in the reactor before HPMA is added;

(iii) Adding further isocyanate component(s) and mixing them with the mixture obtained in step (ii)

(iv) adding a mixture of two or more (meth)acrylates, or adding the two or more (meth)acrylates sequentially,

(v) reacting the resulting mixture optionally in the presence of a catalyst to obtain the urethane di(meth)arylate.

It was shown that the addition to HPMA to HDI, which is already present in the reactor and the premixture of these two components positively influences the crystallization rate.

Preferably, the reaction is carried out in the presence of a catalyst. Possible catalysts are selected from the group consisting of dibutyltin dilaurate (DBTL; CAS 77-58-7), zinc neodecanoate (CAS 27253-29-8), bismuth-2-ethylhexanoate (CAS 72877-97-5), zirconium (IV) acetylacetonate (CAS 17501-44-9), zinc octoate (CAS number: 136-53-8), zinc acetylacetonate (CAS number: 14024-63-6) and zinc acetate (CAS number: 557-34-6), preferably zinc neodecanoate.

Furthermore, a polymerization inhibitor can be added. Possible polymerization inhibitors are selected from the group consisting of 2,6-di-tert-butyl-4-methylphenol (BHT; CAS 128-37-0), hydroquinone r (HOME; methyl ether CAS 150-76-5), hydroquinone (HQ; CAS 123-31-9), phenothiazine (Pheno; CAS 92-84-2), 4-hydroxy-2,2,6,6-tetramethyl-1-piperidinyloxy (4OH-Tempo; CAS 2226-96-2), 4-Methoxy-1-naphthol (CAS number: 84-85-5) and Irganox 1010 (CAS number: 6683-19-8), preferably 2,6-di-tert-butyl-4-methylphenol.

### EXAMPLES

Example 1 shows a representative synthesis for the dimethacrylates according to the present invention.

The following raw materials were used:

GENOMER* 4247 (CAS number: 72869-86-4); IPDI CAS number: 4098-71-9, HDI (CAS number: 822-06-0); H12MDI (CAS number: 5124-30-1); 2,4-TDI (CAS number: 584-84-9); 2,6-TDI (CAS number: 91-08-7); 20% 2,6-TDI+80% 2,4-TDI mix (CAS number: 26471-62-5); MDI (CAS number: 101-68-8); TMXDI (CAS number: 58067-42-8); HEA (CAS number: 818-61-1); HEMA (CAS number: 868-77-9); HPA (CAS number: 4835-90-9); HPMA (CAS number: 27813-02-1); HBA (CAS number: 2478-10-6); HBMA (CAS number: 997-46-6); dibutyltin dilaurate, DBTL (CAS number: 77-58-7); zinc neodecanoate (CAS number: 27253-29-8); bismuth-2-ethylhexanoate (CAS number: 72877-97-5); zirconium (IV) acetylacetonate (CAS number: 17501-44-9); zinc octoate (CAS number: 136-53-8); zinc acetylacetonate (CAS number: 14024-63-6); zinc acetate (CAS number: 557-34-6); 2,6-di-tert-butyl-4-methylphenol, BHT (CAS number: 128-37-0); hydroquinone methyl ether, HQME (CAS number: 150-76-5); hydroquinone, HQ (CAS number: 123-31-9); phenothiazine, Pheno (CAS number: 92-84-2); 4-hydroxy-2,2,6,6-tetramethyl-1-piperidinyloxy, 4OH-Tempo (CAS number: 2226-96-2); 4-Methoxy-1-naphthol (CAS number: 84-85-5); Irganox 1010 (CAS number: 6683-19-8).

The measurements of the parameters mentioned below were conducted according to the following standards:
Elastic Modulus: ISO 527-2
Tensile Strength: ISO 527-2
Elongation: ISO 527-2
Hardness: ISO 868
Viscosity: ISO 3219-1

### Example 1: Synthesis of EX. 13

1 L-four necked flask was charged with 166 g of IPDI and 234 g HDI. The product is stabilized with 0.5 g BHT and catalyzed with 0.5 g zinc neodecanoate. The mixture is stirred at 40° C. A mixture of 138 g HEMA and 460 g HPMA are added dropwise. The exothermic reaction was controlled over the addition speed of the mixture of HEMA and HPMA in order to not exceed 65° C. The reaction can be monitored with FTIR. The identification of the product was carried out by Size exclusion chromatography (SEC): Mn=764 g/mol; Mw=817 g/mol; Mw/Mn=1.071

### Example 2

Table 1: reaction products of several combinations of HDI, IPDI, H12MDI, HEMA, HPMA. In the Table
1 stands for no crystals or only very few crystals
2 stands low tendency to form crystals
3 stands for crystallized

TABLE 1

| | Raw materials | | | | | Viscosity at 25° C. | |
|---|---|---|---|---|---|---|---|
| | HDI | IPDI | H₁₂MDI | HEMA | HPMA | mPas | Cryst. |
| G1 4247 | — | — | — | 100 | — | 10′800 | 1 |
| Ex. 1 | 100 | — | — | 50 | 50 | Solid | 3 |
| Ex. 2 | — | 100 | — | 50 | 50 | >500′000 | 1 |
| Ex. 3 | — | — | 100 | 50 | 50 | >500′000 | 1 |
| Ex. 4 | 35 | 65 | — | 50 | 50 | 185′000 | 1 |
| Ex. 5 | 35 | — | 65 | 50 | 50 | 310′000 | 1 |
| Ex. 6 | 100 | — | — | — | 100 | 5′200 | 3 |
| Ex. 7 | — | 100 | — | — | 100 | >500′000 | 1 |
| Ex. 8 | — | — | 100 | — | 100 | >500′000 | 1 |
| Ex. 9 | 65 | 35 | — | 65 | 35 | 15′500 | 3 |
| Ex. 10 | 65 | — | 35 | 65 | 35 | 21′000 | 3 |
| Ex. 11 | 33 | 33 | 33 | 50 | 50 | 268′000 | 1 |
| Ex. 12 | 65 | 35 | — | 35 | 65 | 20′600 | 2 |
| Ex. 13 | 65 | 35 | — | 25 | 75 | 22′800 | 1-2 |
| Ex. 14 | 65 | — | 35 | — | 100 | 49′000 | 1 |
| Ex. 15 | 65 | 35 | — | 20 | 80 | 23′400 | 1 |
| Ex. 16 | 60 | 40 | — | 25 | 75 | 31′400 | 1 |

### Example 3

Different mechanical properties of different compositions according to Table 1 were measured.

FIG. 1A shows the E-Modulus in MPa of Genomer 4247 (CAS NO. 72869-86-4), EX. 13 (HDI: 65%; IPDI: 35%; HEMA: 35% HPMA: 65%), EX. 18 (HDI: 65%; IPDI: 35%; HEMA: 20%; HPMA: 80%) and EX. 19 (HDI: 60%; H12MDI: 40%; HEMA: 25% HPMA: 75%).

FIG. 1B shows the tensile strength in MPa of Genomer 4247 (CAS NO. 72869-86-4), EX. 13 (HDI: 65%; IPDI: 35%; HEMA: 35% HPMA: 65%), EX. 18 (HDI: 65%; IPDI: 35%; HEMA: 20%; HPMA: 80%) and EX. 19 (HDI: 60%; H12MDI: 40%; HEMA: 25% HPMA: 75%).

FIG. 1C shows the Elongation at break in % of Genomer 4247 (CAS NO. 72869-86-4), EX. 13 (HDI: 65%; IPDI: 35%; HEMA: 35% HPMA: 65%), EX. 18 (HDI: 65%; IPDI: 35%; HEMA: 20%; HPMA: 80%) and EX. 19 (HDI: 60%; H12MDI: 40%; HEMA: 25% HPMA: 75%).

All compositions show very similar mechanical properties compared to TMDI/HEMA (Genomer 4247 supplied by Rahn AG (CAS NO. 72869-86-4)).

### Example 4

Several formulations of different composition according to Table 1 were prepared and characterized before and after curing via irradiation and the final physico-mechanical properties of the materials obtained is always comparable with those given by the standard UDMA.

For the formulations disclosed in Table 3, IBOA is used as diluent, whereas for the formulations disclosed in Table 2 Genomer 1122 and HEMA were used as diluent. The ratio was 60 (oligomer): 25 (Genomer 1122): 15 (HEMA).

Table 2 shows the tensile properties and hardness of materials obtained via radiation curing of formulation containing different urethane (meth)acrylates (via 3D printing (DLP) at the wavelength of 385 nm, layer thickness of 50 μm, irradiation time of 2.2 s per layer. The samples were post cured under UV light for 1 h):

| Compound | | | | |
|---|---|---|---|---|
| of the present | | | | |
| invention:dilu- | | Tensile | | Hard- |
| tent:HEMA 60:25:15 + | Modulus | Strength | Elong. | ness |
| TPO 1 wt. % | (Mpa) | (Mpa) | (%) | (D) |
| Genomer 4247 | 4300 ± 200 | 71 ± 1 | 2.8 ± 0 | 88 ± 0 |
| (CAS NO. | | | | |
| 72869-86-4) | | | | |
| EX. 13 | 4600 ± 800 | 66 ± 24 | 2.5 ± 1.0 | 88 ± 1 |
| EX. 15 | 3420 ± 320 | 84.9 ± 2 | 3.9 ± 0.6 | 86 ± 1 |
| EX. 16 | 3770 ± 400 | 92.1 ± 1.4 | 3.8 ± 0.05 | 87 ± 1 |

Table 3 shows the tensile properties and hardness of materials obtained via radiation curing of formulation containing different urethane (meth)acrylates:

| X:1121y | | | | | |
|---|---|---|---|---|---|
| 50:50 + TPO | | | Tensile | | Hard- |
| 1 wt. % + | Visco | Modulus | Strength | Elong. | ness |
| OB + 0.05% | (mPa · s) | (Mpa) | (Mpa) | (%) | (D) |
| Genomer | 125 | 2505 ± 7 | 55.8 ± 0.4 | 3.5 ± 0.2 | 85 ± 0 |
| 4247 | | | | | |
| (CAS NO. | | | | | |
| 72869-86-4) | | | | | |
| EX. 13 | 150 | 2525 ± 35 | 53.2 ± 0.5 | 3.6 ± 0.3 | 85 ± 0 |
| EX. 15 | 170 | 2580 ± 20 | 62.6± | 3.0 ± 0.3 | 84.0 ± 0 |
| EX. 16 | 169 | 2780± | 54.1± | 2.3 ± 0.2 | 85.0 ± 0 |

### Comparative Example 1

Urethane methacrylate according to precursor of Example 3 of CN 115477912:

A mixture of the (meth)acrylates HEMA, HPA and HPMA with a molar ratio of 1:3:1 was reacted with a mixture of the diisocyanates TDI, HDI and MDI with a molar ratio of 1:2:1. The synthesis carried out at a reaction temperature of 65° C. with DBTL as catalyst and BHT as stabilizer. After 6 h of reaction no residual HEMA, HPA and HPMA could be detected, and the reaction was finished. The isocyanate content of the resulting product was at 13.7%.

| | | Molecular | |
|---|---|---|---|
| | Viscosity | weight | |
| | at 25° C. | (Mw GPC) | Color |
| | [mPas*s] | [g/mol] | [APHA] |
| Comparative | 240′000 | 792 | 31 |
| example 1 | | | |
| Composition | 23′400 | 832 | 12 |
| according to | | | |
| Ex. 15 above | | | |
| GENOMER* 4247 | 10′800 | 903 | 3 |

It is shown that viscosity of comparative example 1 is too high for the intended use in 3D printing.

1. Curable urethane di(meth)acrylate comprising a diisocyanato component and a (meth)acrylate component, characterized in that
i. the diisocyanato component consists of
50 to 75% by weight of 1,6-diisocyanatohexane (HDI),
and at least one further diisocyanate, and
ii. the (meth)acrylate component consists of
50 to 100% by weight of hydroxypropylmethacrylate (HPMA) and
optionally at least one further methacrylate or acrylate component.

2. Urethane di(meth)acrylate according to claim 1, wherein the at least one further diisocyanate is selected from the group consisting of isophorondiisocyanate (IPDI), diisocynatodicyclohexylmethane (H12MDI), toluol-2,4-diisocyanat (TDI), 1,5-diisocyanatopentane (PDI) and tetramethylxylylene diisocyanate (TMXDI) or a mixture thereof.

3. Urethane di(meth)acrylate according to claim 1, wherein the (meth)acrylate component comprises at least one further methacrylate component or acrylate component, preferably selected from the group consisting of hydroxyethyl methacrylate (HEMA), hydroxyethyl acrylate (HEA), hydroxypropyl acrylate (HPA) and hydroxybutyl acrylate (HBA).

4. Urethane di(meth)acrylate according to claim 1, wherein the (meth)acrylate component comprises
60-95% by weight of hydroxypropylmethacrylate (HPMA), 5-40% by weight of hydroxyethyl methacrylate (HEMA), and optionally 0 to 10% by weight of an acrylate component selected from the group consisting of hydroxyethyl acrylate (HEA), hydroxypropyl acrylate (HPA), hydroxybutyl acrylate (HBA) and hydroxybutyl methacrylate (HBMA) or a mixture thereof.

5. Urethane di(meth)acrylate according to claim 1,
wherein the (meth)acrylate component consists of
20-35% by weight of hydroxyethyl methacrylate (HEMA)
65-80% by weight of hydroxypropylmethacrylate (HPMA), preferably
20-30% by weight of hydroxyethyl methacrylate (HEMA)
70-80% by weight of hydroxypropylmethacrylate (HPMA).

6. Urethane di(meth)acrylate according to claim 1, characterized in that the diisocyanato component comprises
60 to 65% by weight of 1,6-diisocyanatohexane (HDI).

7. Urethane di(meth)acrylate according to claim 6, characterized in that the diisocyanato component additionally comprises
35% to 40% by weight of isophorondiisocyanate (IPDI).

8. Urethane di(meth)acrylate according to claim 6, characterized in that the diisocyanato component additionally comprises
30 to 35% by weight of 4,4′-diisocynatodicyclo-hexylmethane (H12MDI).

9. Composition comprising the urethane di(meth)acrylate according to claim 1.

10. Use of the composition according to claim 9 for radiation curing applications or for free radical curing application, preferably for radiation curing applications.

11. Use of urethane di(meth)acrylate according to claim 1 as raw material for the preparation of polymers, adhesives or dental materials.

12. Use according to claim 10 as or for the preparation of dental adhesives, cements or filling composites.

13. Use of the composition according to claim 10 for producing pigmented and non-pigmented paints and varnishes, powder coatings, printing inks, printing plates, adhesives, pressure sensitive adhesives, dental compositions, gel coats, photoresists, electroplating resists, etch resists, both liquid and dry films, solder resists, resists to manufacture color filters, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, color proofing systems, glass fibre cable coatings, screen printing stencils, for producing three-dimensional objects by means of stereo-lithography or material jetting, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board.

14. Method for preparing the urethane di(meth)acrylate according to claim 1 involving the following steps
(i) Preparing the diisocyanato component by mixing
HDI and IPDI and/or H12MDI to obtain a mixture of all diisocyanates, whereby the HDI content is 50 to 75% by weight;
adding either (ii)
i. a hydroxypropylmethacrylate (HPMA), or
ii. mixture of two or more (meth)acrylates, whereby one of said two or more (meth)acrylates present is hydroxypropylmethacrylate (HPMA) constituting a minimum of 50% by weight of the (meth)acrylate content, or
iii. adding the two or more (meth)acrylates sequentially, whereby one of said two or more (meth)acrylates present is hydroxypropylmethacrylate (HPMA) constituting a minimum of 50% weight of the (meth)acrylate content;
(iii) reacting the resulting mixture optionally in the presence of a catalyst to obtain the urethane di(meth)arylate.

15. Method for preparing the urethane di(meth)acrylate according claim 1 involving the following steps
(i) Filling HDI in a reactor
(ii) Adding HPMA to HDI and mixing them together
(iii) Adding further isocyanate component(s) and mixing them with the mixture obtained in step (ii)
(iv) adding a mixture of two or more (meth)acrylates, or adding the two or more (meth)acrylates sequentially,
(v) reacting the resulting mixture optionally in the presence of a catalyst to obtain the urethane di(meth)arylate.
